SEQUENCE LISTING (11 sequences)

SEQ ID NO: 1 - protein, 4 aa, Artificial Sequence
  synthetic peptide
  Xaa is Dbu (3)..(3)
KVXT

SEQ ID NO: 2 - protein, 4 aa, Artificial Sequence
  synthetic peptide
  palmiloylated (1)..(1)
  Xaa is Dbu (3)..(3)
KVXT

SEQ ID NO: 3 - protein, 5 aa, Artificial Sequence
  synthetic peptide
KTTKS

SEQ ID NO: 4 - protein, 5 aa, Artificial Sequence
  synthetic peptide
  palmiloylated (1)..(1)
KTTKS

SEQ ID NO: 5 - protein, 4 aa, Artificial Sequence
  synthetic peptide
GQPR

SEQ ID NO: 6 - protein, 4 aa, Artificial Sequence
  synthetic peptide
  palmiloylated (1)..(1)
GQPR

SEQ ID NO: 7 - protein, 6 aa, Artificial Sequence
  synthetic peptide
VGVAPG

SEQ ID NO: 8 - protein, 6 aa, Artificial Sequence
  synthetic peptide
  palmiloylated (1)..(1)
VGVAPG

SEQ ID NO: 9 - protein, 9 aa, Artificial Sequence
  synthetic peptide
AEDEPLLME

SEQ ID NO: 10 - protein, 9 aa, Artificial Sequence
  synthetic peptide
  palmiloylated (1)..(1)
AEDEPLLME

SEQ ID NO: 11 - protein, 6 aa, Artificial Sequence
  synthetic peptide
  amidation (6)..(6)
EEMQRR

(19) United States
(12) Patent Application Publication
(10) Pub. No.: US 2018/0215784 A1
(43) Pub. Date: Aug. 2, 2018

(54) METHOD FOR PREPARATION OF N-ACYL PEPTIDES, POLYPEPTIDES AND PROTEINS

(71) Applicant: NEODEL TEC LTD., Rehovot (IL)

(72) Inventor: Uri COGAN, Rehovot (IL)

(21) Appl. No.: 15/563,002

(22) PCT Filed: Mar. 28, 2016
(86) PCT No.: PCT/IL2016/050330

§ 371 Date: Sep. 29, 2017

(30) Foreign Application Priority Data
Apr. 1, 2015 (IL) 238101

(51) Int. Cl. C07K 1/107; C07K 14/78; C07K 7/16; C07K 5/103; C07K 7/06

(52) U.S. Cl. CPC C07K 1/1077; C07K 14/78; C07K 7/06; C07K 5/1013; C07K 7/16

(57) ABSTRACT

A method is provided for the preparation of an N-acyl peptide, N-acyl polypeptide or N-acyl protein comprising reacting a peptide, polypeptide or protein with an acyl halide in the presence of the biologically compatible tertiary amine nicotinamide as catalyst, thus obtaining the desired N-acyl peptide, N-acyl polypeptide or N-acyl protein.

### FIELD OF THE INVENTION

The present invention relates to a method for N-acylation of peptides, polypeptides and proteins.

### BACKGROUND OF THE INVENTION

Peptides are naturally occurring substances and participate in a variety of processes in the living organism, such as metabolic, hormonal, reproduction and many other processes.

Recently, many peptides have been produced in suitable host cells by recombinant DNA technology or synthetically by well-established peptide synthesis technology for use in the pharmaceutical and cosmetic industries. However, native peptides as well as analogues thereof tend to exhibit high clearance rates which are unacceptable for many clinical indications where a high plasma concentration of the peptide is required over a prolonged period of time. In addition, many synthetic peptides identified as having biological activity or being potential delivery vectors capable of delivering bioactive agents into the cells, are not cell-permeable and do not cross cell membranes, and thus cannot act intracellularly.

To overcome the problems described above, modifications of peptides and peptide analogs have been proposed that can either influence the clearance rate of the peptides in a favorable direction or to impart the peptide with cell-permeability characteristics. One such modification is the introduction of a lipophilic acyl group into a therapeutic peptide causing a desirable protracted profile of action relative to the non-acylated peptide such as less frequent administration of the therapeutic peptide and, thus, reduction of the amount of peptide to be administered. In other aspects, the acylation of peptides with a lipophilic acyl group results in membrane-permeable peptides capable of entering the cells and exerting their biological activity intracellularly or of delivering bioactive agents into the cells.

Acylation of primary and secondary amines to yield amides is preferably carried out with the respective acyl chlorides. The yield of such reactions can be increased considerably by the addition of a tertiary amine catalyst such as triethylamine, pyridine, 4-dimethylaminopyridine and N-dimethylaminoaniline.

When acylation of the amino group of a peptide is carried out for the purpose of using the acylated peptide in pharmaceutical or cosmetic preparations, traces of the tertiary amine catalyst remaining in the solution reaction are undesirable. A major cost during production of therapeutic peptides is the purification steps required to separate the peptide from impurities, which purification steps are usually performed by chromatography implying expensive chromatography matrices and solvents as well as reduced overall yield.

It would be desirable to design acylation methods for production of acylated peptides for therapeutic and cosmetic uses that would eliminate or minimize the need and the costs for their purification.

### SUMMARY OF THE INVENTION

It has now been found in accordance with the present invention that N-acylation of the terminal amino group of a peptide with acyl chlorides in the presence of nicotinamide as catalyst, leads to a smooth reaction with production of the N-acylated peptide in good yields and simplifies the purification steps, consequently minimizing the cost of the purification of the end product.

The present invention thus relates in certain aspects to a method for the preparation of an N-acyl peptide, N-acyl polypeptide or N-acyl protein comprising reaction of the N-terminal amino group of said peptide, polypeptide or protein with an acyl halide in the presence of nicotinamide as catalyst. It should be understood that, besides the acylation of the terminal amino group, additional acylation of free amino groups present in the original peptide molecule such as epsilon amino groups of lysine, take place too.

### BRIEF DESCRIPTION OF THE FIGURES

FIG. 1 shows that N-octanoyl Gly-Pro (Oct-GP) stimulates pro-collagen synthesis/secretion in human skin fibroblasts compared to the control carrier (0.5% ethanol). Lanes 1-4 and 5-8 represent replicates.

FIG. 2 shows that the relative amounts of pro-collagen produced and secreted into the medium by Oct-GP-treated culture are higher than those of the control. The D/P ratio represents densitometry results (D) standardized by protein quantification (P).

### DETAILED DESCRIPTION OF THE INVENTION

It is the aim of the present invention to provide a method for the preparation of N-acylated peptides, polypeptides and proteins that is very simple and avoids the need, or minimizes the cost, of the purification of the end product. The method comprises the reaction of a peptide, polypeptide or protein with an acyl halide in the presence of nicotinamide as a catalyst.

Nicotinamide, also known as nicotinic amide, niacinamide or vitamin B3, is a biologically compatible tertiary amine. As used herein, the term “biologically compatible tertiary amine” refers to a tertiary amine that is able to be in contact with a living tissue or a living system without producing an adverse effect to said tissue or system. In fact, nicotinamide, the generic name of vitamin B3, is required by the organism in small amounts; therefore, even if small traces of nicotinamide remain in the end product, no harm will be caused by administration of the end product to human beings.

The acyl halide for use in the method of the invention may be an acyl fluoride, chloride or bromide. In certain preferred embodiments, the acyl halide is an acyl chloride.

In certain embodiments, the acyl halide is a saturated or unsaturated fatty acid halide containing 6 to 28, optionally 6 to 20, carbon atoms. In certain embodiments, the acyl halide is an unsaturated fatty acid halide containing 12 to 18 carbon atoms including, but not being limited to, myristoyl, elaidoyl or oleoyl halide.

In certain embodiments, the acyl halide is a saturated fatty acid halide containing 6 to 18 carbon atoms including, but not limited to, hexanoyl (caproyl), octanoyl (caprioyl), lauroyl, palmitoyl or stearoyl halide. In certain embodiments, the acyl halide is the hexanoyl, octanoyl or palmitoyl chloride.

As used herein, the term “peptide” refers to a chemical compound containing from two to 20 amino acid residues.

The term “amino acid” refers herein to both proteinogenic and non-proteinogenic amino acids and includes also analogs and chemical derivatives of said amino acids. “Proteinogenic” amino acids are those encoded or found in the genetic code of any organism and include the natural L-amino acids selected from the 20 common alpha-amino acids found in naturally occurring proteins: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. Some non-alpha proteinogenic amino acids exist in certain organisms such as beta-alanine, gamma-aminobutyric acid (GABA), lambda-aminolevulinic acid and 4-aminobenzoic acid (PABA). D-amino acids such as D-alanine, D-glutamate, D-lysine and D-serine and alpha-amino acids without a hydrogen on the alpha-carbon such as aminoisobutyric acid and dehydroalanine are present in certain organisms.

“Non-proteinogenic” or unnatural amino acids are those not naturally encoded or not found in the genetic code of any organism. Examples of non-proteinogenic natural and unnatural amino acids include, without being limited to, ornithine, citrulline, sarcosine, β-alanine, α-aminoisobutyric acid (Aib), β-amino-n-butyric acid, β-aminoisobutyric acid, γ-aminobutyric acid (GABA), δ-aminolevulinic acid, 4-aminobenzoic acid (PABA), norvaline, α-methylnorvaline, isovaline, norleucine, homocysteine, homoserine, O-methyl-homoserine, O-ethyl-homoserine; α-amino-n-heptanoic acid, 2,4-diaminobutyric acid (Dab), O-methyl-L-tyrosine, L-3-(2-naphthyl)alanine, 3-methyl-phenylalanine, p-ethylthiocarbonyl-L-phenylalanine, p-(3-oxobutanoyl)-L-phenylalanine, O-4-allyl-L-tyrosine, 4-propyl-L-tyrosine, tri-O-acetyl-GlcNAcβ-serine, L-Dopa, fluorinated phenylalanine, isopropyl-L-phenylalanine, p-azido-L-phenylalanine, p-nitro-L-phenylalanine p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, biphenylalanine, p-carboxymethyl-L-phenylalanine, p-cyano-L-phenylalanine, m-cyano-L-phenylalanine, 3-amino-L-tyrosine, 3-nitro-L-tyrosine, bipyridyl alanine, p-(2-amino-1-hydroxyethyl)-L-phenylalanine, and p-isopropyl-thiocarbonyl-L-phenylalanine. Both the L- and D-enantiomers of these unnatural amino acids are included in the present invention.

Peptide “analogs” as used herein are modifications of a peptide in which one or more L-alpha-amino acids are deleted or inserted into the molecule of the peptide, or one or more of the L-alpha-amino acids of the peptide are replaced by the corresponding D-isomer or by a non-proteinogenic or unnatural amino acid residue such as those disclosed above. Peptide “derivatives” as used herein are modifications of a peptide in which one or more L-alpha-amino acids are modified by amidation of the C-terminal carboxyl group or of the additional free carboxyl group of aspartic or glutamic acid; acylation or alkylation of the —OH groups of serine, threonine or tyrosine, of the —SH group of cysteine, threonine or tyrosine, or of the free amino group of lysine; and derivatives of phenylalanine, tyrosine and tryptophan substituted at the phenyl ring.

The term “polypeptide”, as used herein, refers to a long, continuous, and unbranched peptide chain containing 21 amino acid residues or more. A “protein” refers to a macromolecule comprising one or more polypeptide chains arranged in a biologically functional way.

The peptides and polypeptides candidates for acylation according to the present invention may be composed only of proteinogenic amino acids, i.e., amino acids naturally encoded or found in the genetic code of an organism.

In certain embodiments, the peptide or polypeptide is composed of proteinogenic natural amino acids selected from the 20 common alpha-amino acids found in naturally occurring proteins. In certain embodiments, one or more of said 20 natural amino acids may be replaced by the corresponding D-isomer or by a non-proteinogenic natural or unnatural amino acid residue.

The following N-acylated peptides are disclosed in WO 2010/122423 for use in cosmetic compositions: Palm-β-Ala-His-OH, Oleyl-Gly-Gly-OH, Palm-His-D-Phe-Arg-NH₂. Palm-Lys-Val-Lys-OH, Elaidyl-Lys-Phe-Lys-OH. Hexanoyl-Arg-Ala-Nle-NH₂, Palm-Lys-Val-Dab-OH, Palm-Lys-Val-Dab-Thr-OH, Palm-Lys-Thr-Thr-Lys-Ser, Palm-Gly-His-Lys-OH, Palm-Gly-Lys-His-OH, Palm-Gly-Gln-Pro-Arg-OH, Palm-Val-Gly-Val-Ala-Pro-Gly-OH, Palm-Ala-Glu-Asp-Glu-Pro-Leu-Leu-Met-Glu-OH, wherein Palm means palmitoyl and Dab means 2,4-diaminobutyroyl.

Accordingly, in certain embodiments, the following peptides may be acylated by the method of the present invention: β-Ala-His-OH, Gly-Gly-OH, His-D-Phe-Arg-NH₂, Lys-Val-Lys-OH, Lys-Phe-Lys-OH, Arg-Ala-Nle-NH₂, Lys-Val-Dab-OH, Lys-Val-Dab-Thr-OH, Lys-Thr-Thr-Lys-Ser, Gly-His-Lys-OH, Gly-Lys-His-OH, Gly-Gln-Pro-Arg-OH, Val-Gly-Val-Ala-Pro-Gly-OH, and Ala-Glu-Asp-Glu-Pro-Leu-Leu-Met-Glu-OH.

In certain embodiments, the peptides to be acylated according to the present invention are peptides that stimulate collagen synthesis for use in the cosmetic industry and pharmaceutical applications. Collagen, a fibrous protein present in the extracellular matrix of living cells, serves as a key structural component of connective tissue. In the skin, large amounts of collagen are found in the dermis, the inner layer of the skin, where collagen fibers form a supporting mesh responsible for skin's mechanical characteristics such as strength, texture and resilience. With aging, the skin's ability to replace damaged collagen diminishes, leading to wrinkles. Collagen has been used in skin creams for decades. However, new collagen must become a part of the dermis and collagen molecules are, unfortunately, too large to penetrate into the dermis when applied to the surface of the skin. A way to overcome this problem is to stimulate the skin to produce more collagen.

Collagen differs greatly from other proteins as it contains amino acids glycine (Gly), proline (Pro) and hydroxyproline (Hyp) in a concentration that is around 10-20 times higher than in other proteins. These amino acids play an important role in building fibrous tissues and stimulation to promote collagen synthesis has been suggested by supplying these key amino acids or by supplying peptides comprising such key amino acids. These peptides are collagen-stimulating peptides and their use in cosmetic products results in improvement of skin texture, reduction of formation of wrinkles, improvement in the moisture content in skin, in the structure of brittle nails and of hair thickness.

In certain embodiments, the collagen-stimulating peptide is the collagen dipeptide Gly-Pro. In certain other embodiments, the peptide is the collagen tripeptide Gly-Pro-Hyp, which difficulty in passing through the skin barrier due to its hydrophilic moieties may be overcome by its acylation. In certain other embodiments, the peptide is the hydrolyzed form of collagen known as Collagen Peptides, Collagen Hydrolysate, Gelatin Hydrolysate, and Hydrolysed Collagen, a product soluble in cold water that contains all the essential amino acids except tryptophan and which amino acid composition is almost similar to collagen in body.

In certain embodiments, the method of the present invention can also be applied to peptides that in their acylated form are known in the cosmetic industry as palmitoyl oligopeptides, and in which the palmitoyl group is linked to a peptide containing 2 or more amino acids. Examples of such acylated peptides are the dipeptide Acetyl-Dipeptide-1 Cetyl Ester, the tri- and tetrapeptides Palmitoyl tripeptide-3, Palmitoyl Dipeptide-5 Diaminobutyloyl Hydroxythreonine, Palmitoyl Dipeptide-6 Diaminohydroxybutyrate) Palmitoyl Tripeptide-1, Palmitoyl-Tetrapeptide-3, Palmitoyl-Tetrapeptide-7, Palmitoyl tripeptide-8, Palmitoyl pentapeptide-4 (all disclosed in EP 2421886).

In certain other embodiments, the peptides candidates for N-acylation by the method of the invention are peptides which block the secretion of neurotransmitters and induce muscle relaxation, such as the peptide Glu-Glu-Met-Gln-Arg-Arg-NH₂ that in its N-acetyl form is known as acetyl hexapeptide-3 or argireline and is used in anti-wrinkle creams.

In further certain embodiments, the method of the invention can be used for N-acylation of peptides, polypeptides and proteins that are useful for clinical indications, but some of them exhibit high clearance rates that are unacceptable for many clinical indications. Examples of such peptides, polypeptides and proteins include, without being limited to, abarelix, adenosine deaminase, adrenocorticotropic hormone (ACTH), afamelanotide, angiotensin, arginase, arginine deaminase, asparaginase, atosiban, buserelin, calcitonin, carfilzomib, cetrorelix, corticotropin-releasing factor, degarelix, deslorelin, desmopressin, endorphins, enkephalins, enterogastrin, eptifibatide, erythropoietin, exendin, gastric inhibitory peptide, glucagon, glucagon-like peptide-1 (GLP-1), glucagon-like peptide-2 (GLP-2), gondadorelin, goserelin, growth hormone releasing factor, histrelin, hypothalamic releasing factors, icatibant, insulin, insulin-like growth factor-1, insulin-like growth factor-2, interferon, laureotide, leuprolide, nafarelin, octreotide, opioids, oxytocin, parathyroid hormone, pituitary adenylate cyclase-activating peptide (PACAP), prolactin, ribonuclease, secretin, somatomedin, somatostatin, somatotropin, superoxide dismutase, thrombopoietin, thyroid stimulating hormones, triptorelin, vasoactive intestinal peptide (VIP) and analogs thereof, and vasopressin. Introduction of a lipophilic acyl group into the therapeutic peptide causes a desirable protracted profile of action relative to the non-acylated peptide, polypeptide or protein, thus resulting in less frequent administration of the therapeutic product that improves the patient's compliance to the prescribed therapy and reduces the amount to be administered.

In certain embodiments, the method of the present invention for the preparation of the N-acylated peptide, polypeptide or protein comprises the following steps:
(i) melting the tertiary amine nicotinamide at about 130° C.-135° C.;
(ii) adding the acyl halide to the melted tertiary amine while mixing and keeping the temperature of step (i);
(iii) adding the peptide, polypeptide or protein to the mixture of acyl halide and melted tertiary amine of (ii), while mixing and keeping the temperature of step (ii), to form the desired solid N-acylated peptide, polypeptide or protein;
(iv) treating the solid N-acylated peptide, polypeptide or protein obtained in step (iii) with water or an organic solvent; and
(v) obtaining the purified N-acylated peptide, polypeptide or protein.

The preferred solvents used in the method are solvents in which nicotinamide is highly soluble: water 1 g/ml or ethanol 1 g/1.5 ml (Merck Index 13th ed. page 1169, 2001). If it is necessary to remove excessive amounts of nicotinamide, the choice of the solvent in step (iv) will depend on the hydrophilicity/hydrophobicity character of the acylated peptide, polypeptide or protein.

In case of small peptides, in step 3 a highly viscous mixture is obtained that turns into a soft paste to which a large volume of an organic solvent, preferably ethanol, is added. The volume of the solution is reduced by evaporation, the solid that precipitates is discarded, and the solution is further concentrated thus obtaining a purified N-acylated peptide with no trace of the original peptide.

In certain embodiments, the acyl halide is octanoyl chloride, the peptide is Gly-Pro, human oxytocin or tuftsin, and the solvent in step (iv) is ethanol or water.

In certain other embodiments, the acyl halide is palmitoyl chloride, the peptide is Gly-Pro or hydrolyzed collagen peptides and the solvent in step (iv) is ethanol or water

In certain other embodiments, the acyl halide is hexanoyl chloride, the peptide is enkephalin, and the solvent in step (iv) is ethanol or water.

The invention will now be illustrated by the following non-limiting Examples.

### EXAMPLES

### Materials

Peptides of hydrolyzed collagen of average molecular weight (MW) of 1000 were obtained from Asterism Healthcare Plus, Inc. (Japan). SCP 2000, a mixture of hydrolyzed collagen peptides having MW in the range of 500-2000, was obtained from Nitta Gelatin NA Inc. (Morrisville, N.C., USA). Human oxytocin was from Prospec Inc. Tuftsin was obtained from Bachem. Leu-enkephalin was obtained from GenScript Inc. All other materials and reagents were from Sigma-Aldrich.

### Example 1. Preparation of N-Palmitoyl-Gly-Pro

To a sample of nicotinamide (30 gr, 246 mmoles), melted at 130° C., palmitoyl chloride (30 gr, 109 mmoles) was added and mixed. After 10 minutes, the dipeptide Gly-Pro (10 gr; 58 mmoles) was added and mixed while the reaction mixture was kept at 130° C. The highly viscous mixture gradually turned into a soft paste, which was allowed to mix at 130° C. for 3 hours, and then 500 ml of boiling 95% ethanol was added. The solution volume was then reduced to 300 ml by evaporation of part of the ethanol and kept overnight at 4° C. The solid that precipitated in the ethanol was filtered off. The solution was further concentrated by evaporating the ethanol to 200 ml. Thin layer chromatography (TLC) with chloroform/methanol/water (11:8:2) on a silica plate revealed the end product N-palmitoyl-Gly-Pro at Rf=0.9, with no trace of the original dipeptide Gly-Pro (Rf=0.2).

### Example 2. Preparation of N-Octanoyl-Gly-Pro

To nicotinamide (30 gr; 246 mmoles), melted at 130° C., octanoyl chloride (20 gr; 123 mmoles) was added and mixed. After 10 minutes, the dipeptide Gly-Pro (10 gr, 58 mmoles) was added and mixed while the reaction mixture was kept at 130° C. (a sample dissolved in ethanol was taken for TLC). The highly viscous mixture gradually turned into a soft paste, which was allowed to mix and stay at 130° C. for 3 hours, and then 300 ml of boiling 95% ethanol was added and the solution was kept at 4° C. overnight. A solid which was formed was filtered off and the product was collected from the supernatant following evaporation of the ethanol. It was further washed with ethanol and dried. TLC with chloroform/methanol/water (11:8:2) on silica plate revealed the end product at Rf=0.9, with no trace of the starting dipeptide Gly-Pro (Rf=0.2).

### Example 3. Preparation of N-Palmitoyl-Collagen Peptides (Average MW 1000)

3.1 In a first example, a sample of peptides of hydrolyzed collagen of average MW of 1000 (10 gr) was first treated with boiling ethanol (100 ml), followed by boiling acetone (100 ml), to remove traces of water, and then dried. To nicotinamide (8 gr; 66 mmoles), melted at 130° C., palmitoyl chloride (8 gr; 29 mmoles) was added and mixed while at 130° C. After about 10 minutes, 8 gr of the ethanol and acetone treated collagen peptides (approximately 8 mmoles) were added and mixed while at 130° C. The highly viscous mixture gradually turned into a soft, yellow paste which was allowed to mix and stay at 130° C. for 2 hours. 2 ml of water was added to decompose the un-reacted palmitoyl chloride. For estimation of the degree of palmitoylation, a sample of 1 gr from this mixture was first dissolved in 5 ml of 0.2M HCl and then treated twice with 5 ml hexane to extract the un-reacted palmitic acid. The hexane solution was then evaporated and the un-reacted palmitic acid was determined by weighing. The aqueous fraction was then mixed 1:1 with concentrated HCl, to reach approximately 6M, and boiled for 1 hr to hydrolyze the palmitic acid bound to the peptide. It was then followed by hexane extraction and determination of the palmitic acid by weighing, as described above. In this example, we determined 80 mg of free palmitic acid and 24 mg of bound palmitic acid, which corresponded to a mole ratio of 0.3 of bound versus free palmitic acid. Assuming an average molecular weight of 1000, this corresponded to an 84% yield of palmitoylation, whereas complete palmitoylation would correspond to an average molecular weight of about 1200. The calculation is made as follows: from a ratio of 0.3 of bound to free palmitoyl we calculate that 3:(10+3)×29=6.7 mmole palmitoyl were consumed in the acylation. There were 8 mmole peptides assuming MW of 1000. This means: 6.7:8=84% of the peptides were acylated.]

3.2 In a second example, to nicotinamide (8 gr, 66 mmoles), melted at 130° C., palmitoyl chloride (8 gr; 29 mmoles) was added and mixed while at 130° C. After about 10 minutes, 10 gr of the ethanol and acetone treated collagen peptides as described in 3.1 above (approximately 10 mmoles) were added and mixed while at 130° C. The highly viscous mixture gradually turned into a soft, yellow paste which was allowed to mix and stay at 130° C. for 2 hours. Following cooling to room temperature, 2 ml of water was added to decompose the un-reacted palmitoyl chloride. It was then treated twice with 100 ml of ethanol to remove the remains of nicotinamide and free palmitic acid. The residue, which consisted of the modified peptides, was collected by filtration, then dried and weighed, yielding 12.2 gr, namely, addition of 2.2 gr to the original 10 gr of the collagen peptides. Considering the MW of palmitoyl, namely ˜239, this means an extent of palmitoylation of approximately 2.2:2.39, ˜92%. Furthermore, considering the very high solubility of nicotinamide and palmitic acid in ethanol, it follows that these compounds were essentially removed by washing with ethanol.

### Example 4. Preparation of N-Palmitoyl-Collagen Peptides (Average MW 500-2000)

A sample of hydrolyzed collagen peptides, SCP-2000, having MW in the range of 500-2000, was first treated with boiling ethanol (100 ml), followed by of boiling acetone (100 ml) to remove traces of water, and then dried. To nicotinamide (8 gr; 66 mmoles), melted at 130° C., palmitoyl chloride (8 gr; 29 mmoles) was added and mixed while at 130° C. After 10 minutes 8 gr of the ethanol and acetone treated collagen peptides were added and mixed while at 130° C. The highly viscous mixture gradually turned into a soft, yellow paste which was allowed to mix and stay at 130° for 2 hours. 2 ml of water was added to decompose the un-reacted palmitoyl chloride. For estimation of the degree of palmitoylation, a sample of 1 gr from this mixture was first dissolved in 5 ml of 0.2 M HCl and then treated twice with 5 ml hexane to extract the un-reacted palmitic acid. The hexane solution was then evaporated and the un-reacted palmitic acid was determined by weighing. The aqueous fraction was then mixed 1:1 with concentrated HCl, to reach approximately 6 M, and boiled for 1 hr to hydrolyze the palmitic acid bound in the peptide bond. It was then followed by hexane extraction and determination by weighing of the released palmitic acid.

In this example, the ratio of bound versus free palmitic acid, after the termination of the reaction, was found to be 0.4. From a ratio of 0.4 bound to free palmitic acid, we calculate that 4:(10+4)×29=8.28 mmole palmitoyl chloride were consumed for the acylation. From this, assuming that the acylation reaction was nearly completed, we conclude that the average molecular weight of the unreacted collagen hydrolyzate must be slightly below 1000.

### Example 5. Preparation of N-Octanoyl-Human Oxytocin

Human oxytocin (HOT), an octapeptide of MW=1007, has only one site for acylation, the N-terminal alpha amino group. HOT (100 mg; 0.1 mmole) was treated with nicotinamide and octanoyl chloride analogously to the procedure described in Example 1 using the corresponding mole ratios. The acylated peptide was purified by ethanol washing: the solid was treated twice with 2 ml ethanol to remove the highly ethanol soluble nicotinamide, ethyl octanate and octanoic acid. The net weight of the purified N-octanoyl human oxytocin was 114.2 mg, which indicated essentially complete acylation with some residual reactants (complete acylation of the peptide should have yielded 112.5 mg acylated peptide).

### Example 6. Preparation of N-Octanoyl-Tuftsin

Tuftsin, a tetrapeptide of MW=501, has 2 sites which can undergo acylation: the alpha amino of threonine and the epsilon amino of lysine. Tuftsin (100 mg, 0.2 mmole) was treated with nicotinamide and octanoyl chloride with quantities and procedure analogous to those described in Example 2 above. Purification and isolation of the acylated peptide by treatment with 95% ethanol followed the same pathway as for acylated human oxytocin in Example 4 above. The net weight of the acylated peptide was 154.2 mg, indicating essentially complete acylation of both sites, with minor presence of residual reactants (complete octanoylation of both sites should have yielded 150.4 mg).

### Example 7. Preparation of N-Hexanoyl-Enkephalin

Acylation of pentapeptide leu-enkephalin (100 mg) was carried out by reaction with nicotinamide and hexanoyl chloride as described in previous examples. The weight of the ethanol purified N-hexanoyl-peptide was 126.7 mg, indicating again complete acylation with residual impurities of the reactants (complete acylation should have yielded 117.1 mg acylated peptide).

### Example 8. Stimulation of Pro-Collagen Synthesis by N-Octanoyl-Gly-Pro in Cultured Human Skin Fibroblasts

The effect of N-octanoyl-Gly-Pro (also referred to herein as OGP) prepared in Example 2 on intracellular synthesis of pro-collagen, the precursor of collagen, was tested. OGP was applied from a 250 mg/ml stock solution in ethanol. The composition of the solute, estimated by thin layer chromatography, was 80% OGP, 15% nicotinamide and 5% octanoic acid.

The human skin fibroblast cell line CRL1121, obtained from ATCC (American Type Culture Collection), was used for monitoring pro-collagen synthesis. Cells were grown in high glucose-DMEM containing 10% Fetal Calf Serum and antibiotics (streptomycin and penicillin). Confluent cells were suspended and inoculated into wells in 96-well microplates at 7,500 cells/well. At confluency, the cells were placed on fresh medium plus sodium ascorbate (50 μg/ml) for 24 hrs. The cells were then washed with phosphate-buffered saline (PBS) and placed on serum-free medium containing sodium ascorbate, 0.5% ethanol and 50 μM OGP. Controls included cells placed in the same medium, without OGP. After 24 hrs incubation of the experimental and control cells, media were collected, centrifuged, and the proteins were denatured by SDS and heat, and stored at −20° C. for immunoblotting analyses. The cell layers were washed with PBS, dissolved in SDS sample buffer, centrifuged and stored at −20° C. until use.

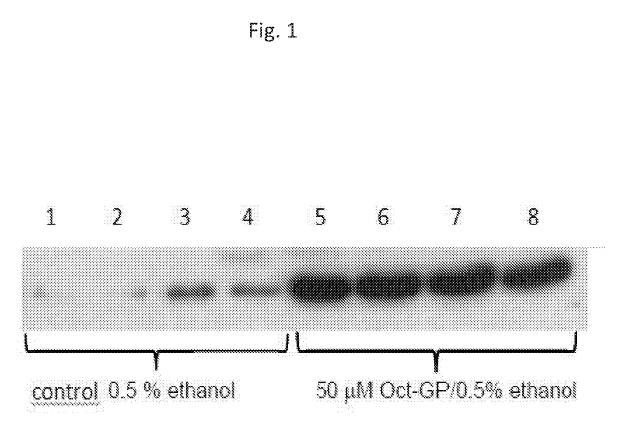

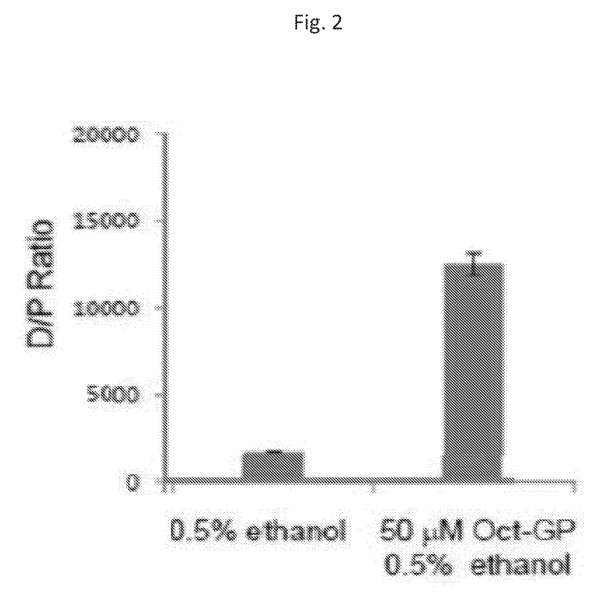

The relative amount of pro-collagen secreted into the medium was determined by immunoblotting with an antibody directed to the C-terminal telopeptide of the α1(I) collagen chain, followed by enhanced chemiluminescence detection and densitometry (Shalitin N, Schlesinger H, Levy, M J, Kessler E, Kessler-Icekson G. Expression of procollagen C-proteinase enhancer in cultured rat heart fibroblasts: Evidence for co-regulation with type I collagen, J. Cell. Biochem. 90:397-407, 2003). Protein concentrations in the cell lysates were determined using the Lowry method and served for standardization. The relative Optical Density of the band corresponding to the (I) chain of pro-collagen was divided by the amount of protein and was expressed as D/P (i.e. band intensity per 1 μg protein). The results of the immunoblotting runs, performed in tetraplicates, and presented in FIG. 1, clearly show that at 50 μM, the acylated peptide OGP greatly enhanced pro-collagen synthesis in the cultured human skin fibroblasts. FIG. 2 presents the relative amounts of the produced and secreted pro-collagen in the OGP-treated culture compared to the control. Using the densitometry readings of lanes 3 and 4 of the control, and standardizing to protein contents (a measure of cell number), the results show that OGP increased pro-collagen synthesis and secretion by at least a factor of 8.3.

1. A method for the preparation of an N-acyl peptide, N-acyl polypeptide or N-acyl protein comprising reacting a peptide, polypeptide or protein with an acyl halide in the presence of the biologically compatible tertiary amine nicotinamide as catalyst, thus obtaining the desired N-acyl peptide, N-acyl polypeptide or N-acyl protein.

2. The method according to claim 1, wherein said acyl halide is a saturated or unsaturated fatty acid halide containing 6 to 28 carbon atoms.

3. The method according to claim 2, wherein said acyl halide is a saturated fatty acid halide containing 6 to 18 carbon atoms.

4. The method according to claim 1, wherein said acyl halide is a fatty acid fluoride, chloride or bromide, preferably a fatty acid chloride.

5. The method according to claim 4, wherein said fatty acid chloride is selected from the group consisting of hexanoyl, octanoyl and palmitoyl chloride.

6. The method according to claim 1, wherein the N-terminal amino group of said peptide, polypeptide or protein is N-acylated.

7. The method according to claim 1, for the preparation of an N-acyl peptide or an analog or derivative thereof.

8. The method according to claim 7, wherein said peptide or analog or derivative thereof has 2 to 20 natural amino acid residues or one or more of said natural amino acid residues have been replaced by the corresponding D-isomer or by a unnatural amino acid residue.

9. The method according to claim 8, wherein said peptide is selected from the group consisting of collagen dipeptide Gly-Pro, human oxytocin, tuftsin and leu-enkephalin.

10. The method according to claim 8, wherein said peptide is selected from group consisting of β-Ala-His-OH, Gly-Gly-OH, His-D-Phe-Arg-NH₂, Lys-Val-Lys-OH, Lys-Phe-Lys-OH, Arg-Ala-Nle-NH₂, Lys-Val-Dab-OH, Lys-Val-Dab-Thr-OH (SEQ ID NO: 1), Dab-Val-Dab-OH, Lys-Thr-Thr-Lys-Ser (SEQ ID NO:3), Gly-His-Lys-OH, Gly-Lys-His-OH, Gly-Gln-Pro-Arg-OH (SEQ ID NO:5), Val-Gly-Val-Ala-Pro-Gly-OH (SEQ ID NO:7), Ala-Glu-Asp-Glu-Pro-Leu-Leu-Met-Glu-OH (SEQ ID NO:9), wherein Dab means 2,4-diaminobutyroyl.

11. The method according to claim 8, wherein said peptide is abarelix, afamelanotide, atosiban, buserelin, carfilzomib, cetrorelix, degarelix, deslorelin, desmopressin, eptifibatide, gondadorelin, goserelin, histrelin, icatibant, lanreotide, leuprolide, nafarelin, octreotide, triptorelin, or vasopressin.

12. The method according to claim 1, for acylation of collagen peptide of average molecular weight of 500-2,000.

13. The method according to claim 1, for the preparation of an N-acylated polypeptide or protein.

14. The method according to claim 13, wherein said polypeptide or protein include adenosine deaminase, adrenocorticotropic hormone (ACTH), angiotensin, arginase, arginine deaminase, asparaginase, calcitonin, corticotropin-releasing factor, endorphins, enkephalins, enterogastrin, erythropoietin, exendin, gastric inhibitory peptide, glucagon, glucagon-like peptide-1 (GLP-1), glucagon-like peptide-2 (GLP-2), growth hormone releasing factor, hypothalamic releasing factors, icatibant, insulin, insulin-like growth factor-1, insulin-like growth factor-2, interferon, opioids, oxytocin, parathyroid hormone, pituitary adenylate cyclase-activating peptide (PACAP), prolactin, ribonuclease, secretin, somatomedin, somatostatin, somatotropin, superoxide dismutase, thrombopoietin, thyroid stimulating hormones, triptorelin, vasoactive intestinal peptide (VIP) and analogs thereof, and vasopressin.

15. The method according to claim 1, said method comprising the steps:
(i) melting the tertiary amine nicotinamide at about 130° C.-135° C.;
(ii) adding the acyl halide to the melted tertiary amine while mixing and keeping the temperature of step (i);
(iii) adding the peptide, polypeptide or protein to (ii) while mixing and keeping the temperature of step (ii) to form the N-acyl peptide;
(iv) treating the solid N-acyl peptide, polypeptide or protein obtained in step (iii) with water or an organic solvent; and
(v) obtaining the purified N-acyl peptide, polypeptide or protein.

16. The method according to claim 15, wherein the organic solvent in step (iv) is ethanol.

17. The method according to claim 2, wherein said saturated or unsaturated fatty acid halide contains 6 to 20 carbon atoms.

18. The method according to claim 3, wherein said saturated fatty acid halide is selected from the group consisting of hexanoyl, octanoyl, myristoyl and palmitoyl halide.

19. The method according to claim 4, wherein said acyl halide is a fatty acid chloride.

20. The method according to claim 6, wherein a further free amino group existing in the peptide, polypeptide or protein molecule is also N-acylated.
